(19) United States
(12) Patent Application Publication
(10) Pub. No.: US 2020/0253685 A1
(43) Pub. Date: Aug. 13, 2020

(54) SINGLE-USE SURGICAL MARKING PEN

(71) Applicants: Jennifer Burns, St. Petersburg, FL (US); Anita Price, St. Petersburg, FL (US)

(72) Inventors: Jennifer Burns, St. Petersburg, FL (US); Anita Price, St. Petersburg, FL (US)

(21) Appl. No.: 16/272,267

(22) Filed: Feb. 11, 2019

(51) Int. Cl. A61B 90/00

(52) U.S. Cl. CPC A61B 90/39; C09D 11/16

(57) ABSTRACT

A single-use surgical marking pen assembly is disclosed. The assembly comprises a housing configured to at least partially receive an elongated member having a first end and a second end. The first end is attached to an ink cartridge terminating in an ink tip while the second end is engaged with a stopper. A plunger end is configured to selectively retract the ink tip into the interior cavity of the housing via the elongated member. A circumferential band is configured to releasably engage with a cap configured to conceal the ink tip when engaged with the circumferential band. A stoppage is positioned on an interior surface of the housing to restrict the movement of the stopper, the restricted movement of the stopper prohibits the extension of the ink tip.

### TECHNICAL FIELD

The embodiments generally relate to marking pens, and more specifically, relate to single-use surgical marking pens.

### BACKGROUND

In the healthcare field, skin marking devices are utilized to delineate boundaries of the area on a body requiring surgical or medical treatment or monitoring, such as to mark incision sites, to identify areas contacted with allergens, and to monitor the state of skin infections. In other industries, skin marking devices are used to monitor individuals in an event space, mark a tattoo to be affixed, or simply for decorating one's skin.

A constant focus within the healthcare industry is the reduction of infection within a care facility. Since at least the early 19^{th} century, caregivers have begun implementing various procedures, starting with simple handwashing techniques, to reduce the transmission rate of infectious agents in the healthcare setting.

Surgical marking pens are a common device used for a variety of purposes. Although simple to use, due to the ink and ink cartridges, care providers are unable to thoroughly sterilize the pens after each use. Despite that present devices may be disposed of after each use, this practice is costly, enticing some to use the pen in multiple marking events and thus increasing the risk of transmitting infection between patients.

### SUMMARY OF THE INVENTION

This summary is provided to introduce a variety of concepts in a simplified form that is further disclosed in the detailed description of the invention. This summary is not intended to identify key or essential inventive concepts of the claimed subject matter, nor is it intended for determining the scope of the claimed subject matter.

The embodiments described herein provide for a single-use surgical marking pen assembly. The assembly comprises a housing configured to at least partially receive an elongated member having a first end and a second end. The first end is attached to an ink cartridge terminating in an ink tip while the second end is engaged with a stopper. An plunger end is configured to selectively retract the ink tip into the interior cavity of the housing via the elongated member. A circumferential band is configured to releasably engage with a cap configured to conceal the ink tip when engaged with the circumferential band. A stoppage is positioned on an interior surface of the housing to restrict the movement of the stopper, the restricted movement of the stopper prohibits the extension of the ink tip.

In one aspect, the single-use surgical marker is produced in an in-use configuration which is defined by the extended position of the ink tip.

In one aspect, the user may press the end to result in the retraction of the ink tip into the interior cavity of the housing.

In one aspect, the single-use surgical marking pen is further comprised of a frustoconical terminal end positioned at a second end of the housing. The frustoconical terminal end may be configured to at least partially protrude from the circumferential band.

In another aspect, the frustoconical terminal end is comprised of a biohazard indicator.

In one aspect, the elongated member is a spring-activated elongated member which may be selectively activated by the plunger end following use of the single-use surgical marking pen.

In one aspect, the circumferential band is comprised of a receiver to releasably engage with the cap via a protrusion. The protrusion and receiver engagement deters the reengagement of the cap with the circumferential band once removed.

### BRIEF DESCRIPTION OF THE DRAWINGS

A complete understanding of the present invention and the advantages and features thereof will be more readily understood by reference to the following detailed description when considered in conjunction with the accompanying drawings wherein:

FIG. 1 illustrates a perspective view of the single-use surgical marking pen, according to some embodiments;

FIG. 2 illustrates an exploded view of the single-use surgical marking pen, according to some embodiments;

FIG. 3 illustrates a perspective view of the single-use surgical marking pen in an in-use configuration, according to some embodiments;

FIG. 4A illustrates a cutaway view of the single-use surgical marking pen with the ink tip in an extended configuration, according to some embodiments;

FIG. 4B illustrates a cutaway view of the single-use surgical marking pen with the ink tip in a retracted configuration, according to some embodiments;

FIG. 5A illustrates a cross-section view of the stopper of the single-use surgical marking pen, according to some embodiments; and

FIG. 5B illustrates a cross-section view of the ink cartridge and spring shaft of the single-use surgical marking pen, according to some embodiments.

### DETAILED DESCRIPTION

The specific details of the single embodiment or variety of embodiments described herein are to the described apparatus. Any specific details of the embodiments are used for demonstration purposes only and not unnecessary limitations or inferences are to be understood therefrom.

Before describing in detail exemplary embodiments, it is noted that the embodiments reside primarily in combinations of components related to the single-use surgical marking pen device.

As used herein, relational terms, such as “first” and “second” and the like, may be used solely to distinguish one entity or element from another entity or element without necessarily requiring or implying any physical or logical relationship or order between such entities or elements.

The embodiments described herein generally relate to skin marking devices including surgical pens configured as a single-use device by restricting the user from using the surgical pen a second time. The single-use surgical pen is configured to mark skin or a similar proteinaceous surface with an ink composition. It is to be understood that the ink composition may be any ink composition known in the arts and may specifically include ink compositions commonly used in the healthcare industry to mark the skin of a patient.

It should be understood that the surgical marking pen of the present embodiments is applicable to marking the skin of both humans and animals, in connection with any diagnostic or therapeutic procedure in which such marking may be helpful.

FIG. 1 shows an exemplary embodiment of the single-use surgical marking pen 100 including a chamber 105 extending between a removable end cap 110 and a push button 125. The removable end-cap 110 is dimensioned to conceal a shaft 120 and an ink tip 115 extending at least partially from the housing 105.

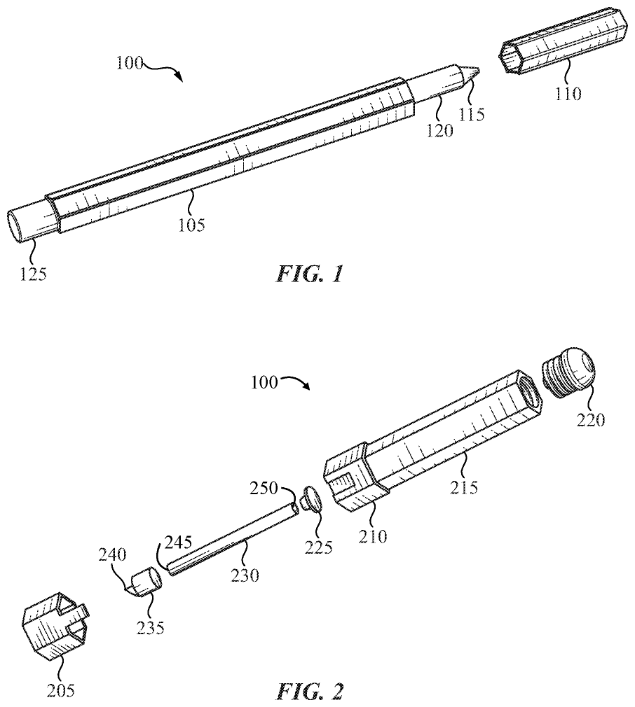

FIG. 2 illustrates an exploded view of the single-use surgical marking pen 100 comprised of a cap 205 configured to releasably engage with a circumferential band 210. The circumferential band 210 is attached, integrally molded, or otherwise affixed to a housing 215. The housing 215 is dimensioned to at least partially receive a plunger end 220, at least one stopper 225, an elongated member 230, an ink cartridge 235, and an ink tip 240.

During production, the single-use surgical marking pen 100 is configured having the ink tip 240 in an extended configuration. A user may remove the cap 205 from the housing 215 by releasing the cap from the circumferential band 210 to expose the ink tip 240. Following use, the user pushes the plunger end 220 to retract the ink tip 240 into the cavity defined by the housing 215 such that the ink tip 240 is unable to perform subsequent use.

To retract the ink tip 240, the user pushes on the plunger end 220 to affect the stopper 225 positioned at an end of the elongated member extending between the stopper 225 and the ink cartridge 235 and ink tip 240. The stopper 225 prohibits the subsequent extension of the ink tip 240, thus configuring the surgical marking pen as a single-use surgical marking pen.

In another embodiment, the single-use surgical marking pen may be produced to permit a single extension and retraction of the ink tip 240 without allowing for subsequent extensions to be performed.

In some embodiments, the elongated member 230 is a spring-activated elongated member affected by the plunger end 220. The elongated member may be comprised of a first end 245 and a second end 250. The first end 245 engages with the ink cartridge 235 terminating in the ink tip 240 while the second end 250 is engaged with the stopper 225.

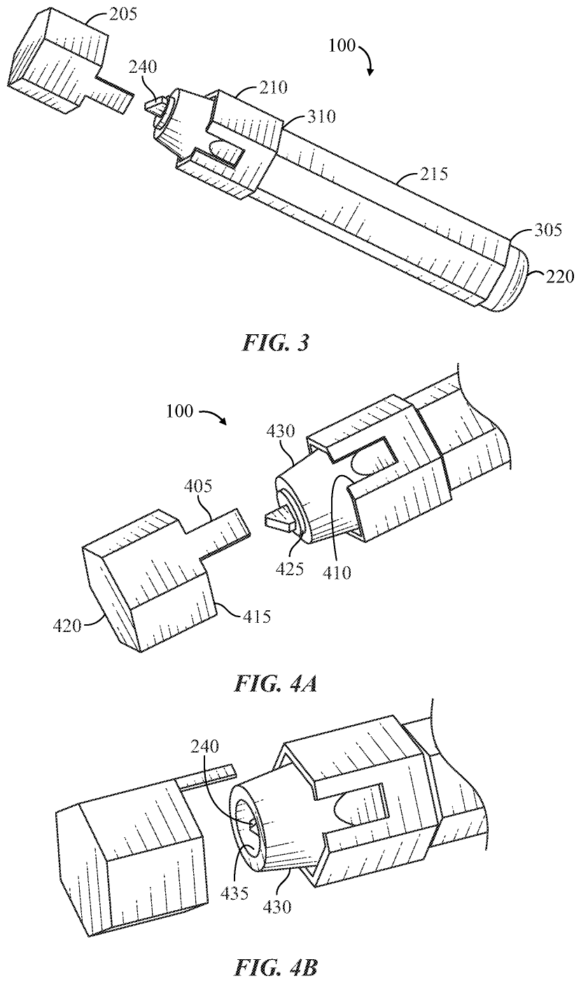

FIG. 3 illustrates the single-use surgical marking pen 100 in an in-use and assembled configuration. The cap 205 is removed from the circumferential band 210 to expose the ink tip 240 to permit the user to mark the skin of a patient or a similar proteinaceous surface. The housing 215 is comprised of a first end 305 configured to engage with the plunger end 220, and a second end 310 comprised of the circumferential band 210.

FIG. 4A illustrates a cutaway view of the single-use surgical marking pen in an in-use configuration having the cap removed from the circumferential band to expose the ink tip. The cap is comprised of a protrusion 405 positioned on a first side 415 of the cap which is configured to mate with a receiver 410 on the circumferential band. The protrusion 405 and receiver 410 deter reattachment of the cap by requiring the protrusion 405 and the receiver 410 to be aligned to permit reattachment of the cap. A second side 420 of the cap is configured to enclose the ink tip and interior components of the single-use surgical marking pen following production or upon the selective reattachment of the cap. The ink cartridge 425 is configured to be received within an interior of a terminal end 430 of the housing. In some embodiments, the terminal end 430 is frustoconical and may include an indicator such as a biohazard indicator or color marking to alert the user that the single-use surgical marking pen has been used or is in use.

FIG. 4B illustrates a cutaway view of the single-use surgical marking pen in a retracted configuration following the use. As illustrated, the ink tip 240 is retracted within an interior cavity 435 of the frustoconical terminal end 430 such that the ink tip 240 is unable to contact the skin of a patient or another surface. To contain infectious agents potentially present on the ink tip 240 or the frustoconical terminal end 430, the user may selectively replace the cap before disposing of the single-use surgical marking pen.

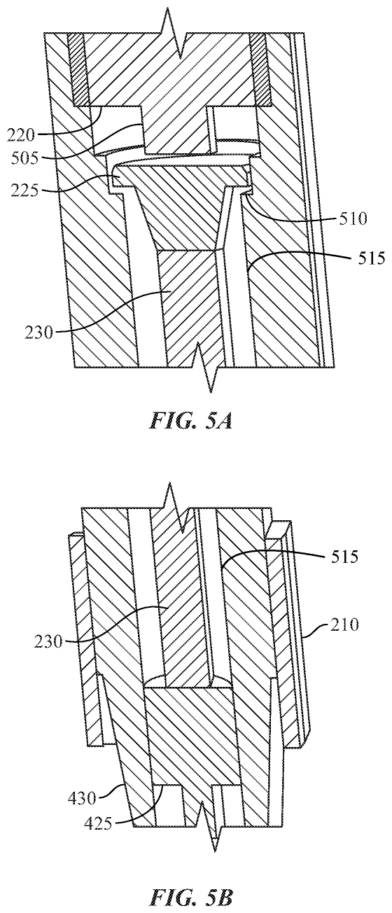

FIG. 5A and FIG. 5B illustrate cross-sectional views of the housing and internal components of the single-use surgical marking pen including the plunger end 220, the stopper 225, the elongated member 230, and the ink cartridge 425. Following use, the user selectively pushes the plunger end 220 to engage a one-way mechanical stop to restrict the ink tip from re-extending, thus restricting subsequent use of the surgical marking pen. A member 505 extends from a bottom of the plunger end 220 to affect the stopper 225. A stoppage 510 is positioned on the interior surface 515 of the housing. The stoppage 510 defines a interior circumference which is less than the circumference of the top portion 520 of the stopper 225. FIG. 5B illustrates the ink cartridge 425 disposed within the frustoconical terminal end 430. The elongated member 430 is affixed to the ink cartridge to retract the ink cartridge 425 following use. The ink cartridge 425 slidingly engages with the interior surface 515 of the housing which is at least partially encapsulated by the circumferential band 210.

Many different embodiments have been disclosed herein, in connection with the above description and the drawings. It will be understood that it would be unduly repetitious and obfuscating to literally describe and illustrate every combination and subcombination of these embodiments. Accordingly, ail embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A single-use surgical marking pen assembly, comprising:
a housing configured to at least partially receive an elongated member including a first end and a second end, the first end attached to an ink cartridge terminating in an ink tip, the second end engaged with a stopper;
a plunger end configured to selectively retract the ink tip into the interior cavity of the housing via the elongated member;
a circumferential band configured to releasably engage with a cap, the cap configured to conceal the ink tip when engaged with the circumferential band; and
a stoppage positioned on an interior surface of the housing to restrict the movement of the stopper, the restricted movement of the stopper prohibits the extension of the ink tip.

2. The single-use surgical marking pen assembly of claim 1, wherein the single-use surgical marker is produced in an in-use configuration, the in-use configuration defined by the extended position of the ink tip.

3. The single-use surgical marking pen assembly of claim 2, wherein pressing the plunger end retracts the ink tip into the interior cavity of the housing.

4. The single-use surgical marking pen assembly of claim 3, further comprising a frustoconical terminal end positioned at a second end of the housing and at least partially protruding from the circumferential band.

5. The single-use surgical marking pen of claim 4, wherein the frustoconical terminal end is comprised of a biohazard indicator.

6. The single-use surgical marking pen of claim 1, wherein the elongated member is a spring-activated elongated member.

7. The single-use surgical marking pen of claim 6, wherein the spring activated elongated member is selectively activated by the plunger end following use of the single-use surgical marking pen.

8. The single-use surgical marking pen of claim 1, wherein the circumferential band is comprised of a receiver to releasably engage with the cap via a protrusion adapted to deter reengagement of the cap with the circumferential band once removed.

9. A single-use surgical marking pen assembly, comprising:
a housing configured to at least partially receive an elongated member including a first end and a second end, the first end attached to an ink cartridge terminating in an ink tip provided in an in-use configuration having the ink tip in an extended position to contact a proteinaceous surface, the second end engaged with a stopper;
a plunger end configured to selectively retract the ink tip into the interior cavity of the housing via the elongated member when pressed;
a circumferential band configured to releasably engage with a cap, the cap configured to conceal the ink tip when engaged with the circumferential band; and
a stoppage integrally molded to an interior surface of the housing, the stoppage configured to restrict the movement of the stopper, the restricted movement of the stopper prohibits the extension of the ink tip.

10. The single-use surgical marking pen of claim 9, wherein the stoppage defines an interior circumference of the housing, wherein the interior circumference of the housing is less than a circumference of the stopper.

11. The single-use surgical marking pen assembly of claim 9, further comprising a frustoconical terminal end positioned at a second end of the housing and at least partially protruding from the circumferential band.

12. The single-use surgical marking pen assembly of claim 11, wherein the frustoconical terminal end is comprised of a biohazard indicator or a biohazard indicating color.

13. The single-use surgical marking pen assembly of claim 11, wherein the frustoconical terminal end is at least partially received by the circumferential band.

14. The single-use surgical marking pen assembly of claim 11, wherein the frustoconical terminal end and the ink tip are encapsulated between the circumferential band and the cap when the cap is engaged with the circumferential band.

15. The single-use surgical marking pen assembly of claim 14, wherein the cap is releasably engageable with the circumferential band in an in-use configuration and when the ink tip is in a retracted configuration.

16. The single-use surgical marking pen of claim 15, wherein the circumferential band is comprised of a receiver to releasably engage with the cap via a protrusion adapted to deter reengagement of the cap with the circumferential band once removed.

17. A single-use surgical marking pen assembly, comprising:
a surgical marking pen provided in an in-use configuration defined by an ink tip extended from a frustoconical terminal end of a housing, the ink tip configured to contact and mark a proteinaceous surface;
an elongated member at least partially received by the housing, the elongated member comprised of a first end and a second end, the first end is attached to an ink cartridge terminating in the ink tip, the second end engaged with a stopper having a circumference greater than an interior circumference of the housing defining a stoppage; and
a plunger end configured to selectively retract the ink tip into an interior cavity of the housing via the elongated member, the stoppage configured to prohibit the extension of the ink tip.

18. The single-use surgical marking pen assembly of claim 17, further comprising a circumferential band configured to releasably engage with a cap, the cap configured to conceal the ink tip when engaged with the circumferential band.

19. The single-use surgical marking pen assembly of claim 17, wherein the elongated member is a spring-activated elongated member.

20. The single-use surgical marking pen assembly of claim 17, wherein the ink tip retains an ink composition safe for contact to the proteinaceous surface.
